(19) United States
(12) Patent Application Publication
(10) Pub. No.: US 2006/0223074 A1
(43) Pub. Date: Oct. 5, 2006

(54) Spotting compositions and methods of use thereof

(76) Inventors: Thomas A. Bunch, Painted Post, NY (US); Katie M. Ruocco, Frederick, MD (US)

Correspondence Address: CORNING INCORPORATED, SP-TI-3-1, CORNING, NY, 14831, US

(21) Appl. No.: 11/100,259

(22) Filed: Apr. 5, 2005

(51) Int. Cl. C12Q 1/68; G01N 33/53; C12N 1/08

(52) U.S. Cl. 435/6; 435/7.1; 435/270; 427/2.11

(57) ABSTRACT

Described herein are spotting solutions and methods of use thereof.

### BACKGROUND

Hybridization is widely used to test for the presence of a nucleic acid sequence that is complementary to a probe moiety. In many cases, this provides a simple, fast, and inexpensive alternative to conventional sequencing methods. Hybridization does not require nucleic acid cloning and purification, carrying out base-specific reactions, or tedious electrophoretic separations. Hybridization of oligonucleotide probes has been successfully used for various purposes, such as analysis of genetic polymorphisms, diagnosis of genetic diseases, cancer diagnostics, detection of viral and microbial pathogens, screening of clones, genome mapping and ordering of fragment libraries.

In heterogeneous assays, nucleic acid arrays can comprise a number of individual oligonucleotide species tethered to the surface of a solid support in a regular pattern, each species in a different area, so that the location of each oligonucleotide is known. An array can contain a chosen collection of oligonucleotides (e.g., probes specific for all known clinically important pathogens or specific for all known clinically important pathogens or specific for all known sequence markers of genetic diseases). Such an array can satisfy the needs of a diagnostic laboratory. Alternatively, an array can contain all possible oligonucleotides of a given length n. Hybridization of a nucleic acid with such a comprehensive array results in a list of all its constituent n-mers, which can be used for a number of assays. Examples include: for unambiguous gene identification (e.g., in forensic studies), for determination of unknown gene variants and mutations (including the sequencing of related genomes once the sequence of one of them is known), for overlapping clones, and for checking sequences determined by conventional methods. Finally, surveying the n-mers by hybridization to a comprehensive array can provide sufficient information to determine the sequence of a totally unknown nucleic acid.

An oligonucleotide array can be prepared by synthesizing all the oligonucleotides, in parallel, directly on the support, employing the methods of solid-phase chemical synthesis in combination with site-directing masks, such as described in U.S. Pat. No. 5,510,270. Four masks with non-overlapping windows and four coupling reactions are required to increase the length of tethered oligonucleotides by one. In each subsequent round of synthesis, a different set of four masks is used, and this determines the unique sequence of the oligonucleotides synthesized in each particular area. Using an efficient photolithographic technique, miniature arrays containing as many as 10⁵ individual oligonucleotides per cm² of area have been demonstrated.

Another technique for creating oligonucleotide arrays involves precise drop deposition using a piezoelectric pump, such as described in U.S. Pat. No. 5,474,796. A piezoelectric pump delivers minute volumes of liquid to a substrate surface. The pump design is very similar to the pumps used in ink jet printing. This picopump is capable of delivering a 50 micron-diameter (about 65 picoliter) droplets at up to 3,000 Hz and can accurately hit a 250 micron target. As an illustration, the pump unit can be assembled with five nozzles array heads, one for each of the four nucleotides and a fifth for delivering, activating agent for coupling. The pump unit remains stationary while droplets are fired downward at a moving array plate. When energized, a microdroplet is ejected from the pump and deposited on the array plate at a functionalized binding site. Different oligonucleotides are synthesized at each individual binding site based on the microdrop deposition sequence.

A popular method for creating high-density arrays uses pins, which are dipped into solutions of biological sample fluids and then touched to a surface. For example, a nucleic acid (e.g., oligonucleotides or DNA) is typically solubilized in an aqueous medium (sometimes referred to as a “printing ink” or “ink”) that contains salts, which are used as components of buffers that are compatible with biological macromolecules. A 3×SSC (450 mM sodium chloride and 45 mM sodium citrate) is a standard concentration for printing inks. See, e.g., U.S. Pat. No. 5,807,522.

Use of SSC-containing inks, however, can be problematic. The first problem encountered in manufacturing DNA arrays using a 3×SSC ink is that the rate of evaporation of the aqueous medium is very high compared to the time required to print multiple slides. This is a major obstacle to scaling up the manufacturing process. Additionally, the 3×SSC ink is not only incapable of printing the required number of slides, but also the quality and performance of arrays printed vary due to the evaporation of aqueous medium, which results in a rapidly changing concentration of DNA.

It is also desirable to have a spotting solution that produces a consistent spot morphology and concentration of selected biomolecule. Moreover, it would be advantageous if the spotting solution can be used across a variety of printers and produce little to no autofluorescence. For example, when the biomolecule is DNA, the autofluorescence can be construed as the expression of a gene after DNA hybridization. The spotting solutions described herein address these needs that are lacking with existing spotting solutions.

### SUMMARY

Described herein are spotting solutions and methods of use thereof. The advantages of the materials, methods, and articles described herein will be set forth in part in the description which follows, or may be learned by practice of the aspects described below. The advantages described below will be realized and attained by means of the elements and combinations particularly pointed out in the appended claims. It is to be understood that both the foregoing general description and the following detailed description are exemplary and explanatory only and are not restrictive.

### BRIEF DESCRIPTION OF FIGURES

The accompanying Figures, which are incorporated in and constitute a part of this specification, illustrate several aspects described below.

FIG. 1 shows the hybridization of test oligo arrays printed with Point Tech pins.

FIG. 2 shows that a spotting solution of the invention does not produce a detectable signal when compared to a commercially available spotting solution.

FIG. 3 shows oligos spotted with a solution of the invention followed by hybridization with cDNA and scanned with Cy3/Cy5 PMT showed no irregularities.

FIG. 4 shows the ability of arrays produced from a spotting solution of the invention can be stored at least for four months.

### DETAILED DESCRIPTION

Before the present materials, articles, and/or methods are disclosed and described, it is to be understood that the aspects described below are not limited to specific compounds, synthetic methods, or uses as such may, of course, vary. It is also to be understood that the terminology used herein is for the purpose of describing particular aspects only and is not intended to be limiting.

In this specification and in the claims that follow, reference will be made to a number of terms that shall be defined to have the following meanings:

Throughout this specification, unless the context requires otherwise, the word “comprise,” or variations such as “comprises” or “comprising,” will be understood to imply the inclusion of a stated integer or step or group of integers or steps but not the exclusion of any other integer or step or group of integers or steps.

It must be noted that, as used in the specification and the appended claims, the singular forms “a,” “an” and “the” include plural referents unless the context clearly dictates otherwise. Thus, for example, reference to “a pharmaceutical carrier” includes mixtures of two or more such carriers, and the like.

“Optional” or “optionally” means that the subsequently described event or circumstance can or cannot occur, and that the description includes instances where the event or circumstance occurs and instances where it does not.

Ranges can be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed, then “less than or equal to 10” as well as “greater than or equal to 10” is also disclosed. It is also understood that throughout the application, data is provided in a number of different formats, and that this data, represents endpoints and starting points, and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Disclosed are compounds, compositions, and components that can be used for, can be used in conjunction with, can be used in preparation for, or are products of the disclosed methods and compositions. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a number of different polymers and biomolecules are disclosed and discussed, each and every combination and permutation of the polymer and biomolecule are specifically contemplated unless specifically indicated to the contrary. Thus, if a class of molecules A, B, and C are disclosed as well as a class of molecules D, E, and F and an example of a combination molecule, A-D is disclosed, then even if each is not individually recited, each is individually and collectively contemplated. Thus, in this example, each of the combinations A-E, A-F, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the sub-group of A-E, B-F, and C-E are specifically contemplated and should be considered disclosed from disclosure of A, B, and C; D, E, and F; and the example combination A-D. This concept applies to all aspects of this disclosure including, but not limited to, steps in methods of making and using the disclosed compositions. Thus, if there are a variety of additional steps that can be performed it is understood that each of these additional steps can be performed with any specific embodiment or combination of embodiments of the disclosed methods, and that each such combination is specifically contemplated and should be considered disclosed.

Described herein are spotting solutions for biomolecules. In one aspect, the solution comprises (a) an alkylene diol; (b) a betaine; (c) a detergent; and (d) a salt. Each component present in the solution will be discussed in detail below.

The term “alkylene diol” as used herein is any compound that possesses two hydroxyl groups and at least one CH₂ group. The alkylene diol can be branched or straight chain. In one aspect, the alkylene diol comprises the formula HO(CH₂)ₙOH, wherein n is an integer of from 1, 2, 3, 4, 5, 6, 7, 8, 9, 10, 11, 12, 13, 14, 15, 16, 17, 18, 19, 20, 21, 22, 23, 24, or 25. In one aspect, the alkylene diol comprises a straight chain compound such as, for example, methylene glycol, ethylene glycol, propylene glycol, butylene glycol, or a mixture thereof. In another aspect, the alkylene diol comprises a branched compound such as, for example, isopropyl diol, isobutyl- and sec-butyl diol, neopentyl diol, and the like. In one aspect, the alkylene diol is from 30 to 70% by volume of the composition. In another aspect, the alkylene diol is 30%, 35%, 40%, 45%, 50%, 55%, 60%, 65%, or 70% by volume of the composition, wherein any value can form a lower and upper endpoint. In one aspect, the alkylene diol is from 40 to 60% by volume of the composition.

Betaines useful herein are generally zwitterionic compounds; however, they can exist in salt forms as well. The betaines disclosed in U.S. Pat. Nos. 6,852,816; 6,846,795; 6,846,352; and 6,849,426, which are incorporated by reference, can be used herein. In one aspect, the betaine comprises an alkyl betaine, a sulfobetaine, an amidobetaine, or a phosphobetaine.

The preparation of such compounds is described, for example, in Fernley, J. Am. Oil Chem. Soc. 55, 98-103 (1978) and U.S. Pat. No. 3,280,179. The sulfobetaines disclosed in U.S. Pat. No. 6,849,426 can also be used herein. In one aspect, the sulfobetaine comprises the formula (R¹)(R²)(R³)N⁺—(CH₂)_{O}—SO₃⁻, wherein R¹, R², and R³ are, independently, hydrogen or an alkyl group, and o is from 1 to 25, wherein the betaine is a neutral compound or the salt thereof. In one aspect, N-tetradecyl-N,N-dimethyl-3-ammonio-1-propanesulfonate, which is commercially available from Calbiochem-Behring Corporation under the trademark ZWITTERGENT 3-14, can be used as the betaine. In another aspect, the sulfobetaine can be 3-(N,N-dimethyl-N-acylamidopropylammonio)-2-hydroxy-propane-1-sulfonates, wherein the acyl group is derived from tallow fatty alcohol or coconut fatty alcohol. In another aspect, the sulfobetaine can be N-cocoamido-propyl-N,N-dimethyl-N-2-hydroxypropyl sulfobetaine. An example of this is LONZAINE CS, which is commercially available from Lonza, Inc., Fair Lawn, N.J.

In one aspect, the phosphobetaine comprises the formula (R¹)(R²)(R³)N⁺—(CH₂)ₚ—OPO₃⁻, wherein R¹, R², and R³ are, independently, hydrogen or an alkyl group, and p is from 1 to 25, wherein the betaine is a neutral compound or the salt thereof. The phosphobetaines disclosed in U.S. Pat. No. 6,852,816 are useful herein.

In another aspect, the betaine can be lauryldimethylaminoacetic acid betaine, 2-alkyl-N-carboxymethyl-N-hydroxyethylimidazolinium betaine, coconut oil fatty acid amide propyldimethylaminoacetic acid betaine, polyoctylpolyaminoethylglycine, and imidazoline derivatives. In another aspect, the betaine can be a C₁₂-C₁₈ dimethyl-ammonio hexanoate or a C₁₀-C₁₈ acylamidopropane (or ethane) dimethyl (or diethyl) betaine.

In one aspect, the betaine comprises the formula (R¹)(R²)(R³)N⁺—CH₂—COO⁻, wherein R¹, R², and R³ are, independently, hydrogen or an alkyl group, wherein the betaine is a neutral compound or the salt thereof. In one aspect, R¹, R², and R³ are, independently, methyl, ethyl, propyl, butyl, or pentyl. In another aspect, R¹, R², and R³ are methyl.

Detergents useful herein include, but are not limited to, a surfactant. A “surfactant” as used herein is a molecule composed of hydrophilic and hydrophobic groups (i.e., an amphiphile). Suitable surfactants can be generally classified as ionic (anionic/cationic/dipolar) and nonionic. In one aspect, polymeric surfactants, natural surfactants, silicon surfactants, fluorinated surfactants, oligomeric surfactants, dimeric surfactants, and the like, are suitable for the compositions and methods disclosed herein. In one aspect, the surfactants disclosed in U.S. Pat. No. 6,849,426, which is incorporated by reference in its entirety, can be used herein.

In one aspect, the detergent comprises an anionic surfactant. Any anionic surfactants known in the art can be used herein. In one aspect, the anionic surfactant comprises an alkyl aryl sulfonate, an alkyl sulfate, or sulfated oxyethylated alkyl phenol. In another aspect, the anionic surfactant can be an alkylbenzene sulfonate (detergent), a fatty acid based surfactant, a lauryl sulfate (e.g., a foaming agent), a di-alkyl sulfosuccinate (e.g., a wetting agent), a lignosulfonate (e.g., a dispersant), and the like, including mixtures thereof. In other examples, linear alkylbenzene sulphonic acid, sodium lauryl ether sulphate, alpha olefin sulphonates, phosphate esters, sodium sulphosuccinates, hydrotropes, and the like, including mixtures thereof, can be used. In one aspect, the anionic surfactant comprises sodium dodecylbenzene sulfonate, sodium decylbenzene sulfonate, ammonium methyl dodecylbenzene sulfonate, ammonium dodecylbenzene sulfonate, sodium octadecylbenzene sulfonate, sodium nonylbenzene sulfonate, sodium dodecylnaphthalene sulfonate, sodium hetadecylbenzene sulfonate, potassium eicososyl naphthalene sulfonate, ethylamine undecylnaphthalene sulfonate, sodium docosylnaphthalene sulfonate, sodium octadecyl sulfate, sodium hexadecyl sulfate, sodium dodecyl sulfate, sodium nonyl sulfate, ammonium decyl sulfate, potassium tetradecyl sulfate, diethanolamino octyl sulfate, triethanolamine octadecyl sulfate, amrnmonium nonyl sulfate, ammonium nonylphenoxyl tetraethylenoxy sulfate, sodium dodecylphenoxy triethyleneoxy sulfate, ethanolamine decylphenoxy tetraethyleneoxy sulfate, or potassium octylphenoxy triethyleneoxy sulfate.

In one aspect, the detergent comprises a nonionic surfactant. Any nonionic surfactant can be used. Suitable nonionic surfactants do not ionize in aqueous solution, because their hydrophilic group is of a non-dissociable type, such as alcohol, phenol, ether, ester, or amide. They can be classified as ethers (e.g., polyhydric alcohols such as glycerin, sorbitole, sucrose, etc.), fatty acid esters (e.g., glycerin fatty acid ester, sorbitan fatty acid ester, sucrose fatty acid ester, etc.), esters (e.g., compounds made by applying, for example, ethylene oxide to a material having hydroxyl radicals such as high alcohol, alkyl-phenol, and the like), ether/esters (e.g., compounds made by applying, for example, the ethylene oxide to the fatty acid or polyhydric alcohol fatty acid ester, having both ester bond and ether bond in the molecule), and other types (e.g., the fatty acid alkanol-amide type or the alkylpolyglyceride type). Other suitable examples of nonionic surfactants can include, but are not limited to, alcohol ethoxylates and alkyl phenol ethyoxylates, fatty amine oxides, alkanolamides, ethylene oxide/propylene oxide block copolymers, alkyl amine ethoxylates, tigercol lubricants, etc. In one aspect, the nonionic surfactant comprises the condensation product between ethylene oxide or propylene oxide with the propylene glycol, ethylene diamine, diethylene glycol, dodecyl phenol, nonyl phenol, tetradecyl alcohol, N-octadecyl diethanolamide, N-dodecyl monoethanolamide, polyoxyethylene sorbitan monooleate, or polyoxyethylene sorbitan monolaurate.

In another aspect, the detergent comprises a cationic surfactant. Any cationic surfactant known in the art can be used herein. Suitable cationic surfactants included, but are not limited to, quaternary ammonium compounds, imidazolines, etc. Such cationic surfactants can be obtained commercially or can be prepared by methods known in the art. In one aspect, the cationic surfactant comprises ethyl-dimethylstearyl ammonium chloride, benzyl-dimethyl-stearyl ammonium chloride, benzyldimethyl-stearyl ammonium chloride, trimethyl stearyl ammonium chloride, trimethylcetyl ammonium bromide, dimethylethyl dilaurylammonium chloride, dimethyl-propyl-myristyl ammonium chloride, or the corresponding methosulfate or acetate.

Other examples of suitable surfactant include natural surfactants, which can have their source from plant or animal organs. In another example, a bolaform surfactant can be used. A bolaform surfactant is a surfactant that has two hydrophilic head groups at opposite ends of a hydrophobic tail. In another aspect, the detergent can be Tween-20 or Triton x-100.

In one aspect, the detergent comprises an organic acid or the salt thereof. Examples of organic acids useful herein include saturated or unsaturated fatty acids. In one aspect, the organic acid comprises the formula CH₃(CH₂)ₘCO₂H, wherein m is 1, 2, 3, 4, 5, 6, 7, 8, 9, 10, 11, 12, 13, 14, 15, 16, 17, 18, 19, 20, 21, 22, 23, 24, or 25, or the salt thereof. In another aspect, the detergent comprises hexanoic acid, heptenoic acid, octanoic acid, nonanoic acid, decanoic acid, or the salt thereof. In one aspect, when the detergent comprises an organic acid such as, for example, octanoic acid, the amount of organic acid can be 0.005%, 0.01%, 0.02%, 0.03%, 0.04%, 0.05%, 0.06%, 0.07%, 0.08%, 0.09%, 0.10%, 0.15%, 0.20%, or 0.25% by volume of the composition, where nay value can form a lower and upper endpoint of the concentration range. In one aspect, the amount of organic acid is from 0.05% to 0.10% by volume of the composition.

It is contemplated that mixtures of surfactants can also be used in the compositions and methods disclosed herein.

It is contemplated that the salt present in the compositions described herein can be an organic salt, an inorganic salt, or a mixture thereof. In one aspect, the organic salt comprises a citrate. In another aspect, the inorganic salt comprises NaCl, KCl, MgCl₂, LiCl, or a mixture thereof. In another aspect, the salt comprises a mixture of NaCl and sodium citrate.

The compositions described herein can be prepared by admixing an alkylene diol, a betaine; a detergent; and a salt. The term “admixing” is defined as mixing two or more components together. Depending upon the components to be admixed, there may or may not be a chemical or physical interaction between two or more components. The order the components can be admixed with each other can vary depending upon the selection of starting materials. The components can be admixed using techniques known in the art such as, for example, stirring, shaking, or homogenizing. Likewise, the amounts of each component can also vary, and will be determined by the selection of each component and biomolecule as well as the technique for depositing the biomolecule on the support. In one aspect, water is used as a solvent for the spotting composition.

The pH of the composition can also vary depending upon the selection of starting materials and the biomolecule to be spotted, which can be readily determined by one of ordinary skill in the art. In one aspect, the spotting composition comprises an alkaline pH. In another aspect, the pH is greater than 8.5. In a further aspect, the pH is 8.5, 9.0. 9.5, 10.0, 10.5, 11.0, 11.5, 12.0, 12.5, or 13, where any pH value can form a lower and upper end-point for a pH range. The pH of the spotting composition can be adjusted by adding bases such as, for example, hydroxides, carbonates, phosphates, and the like.

In one aspect, the alkylene diol is ethylene glycol, the betaine is Me₃N⁺—CH₂—COO⁻, the detergent is octanoic acid, and the salt is NaCl and sodium citrate. In this aspect, water can be present as the solvent and the composition has a pH greater than 8.5.

The spotting solutions described herein are used to spot biomolecules on the surface of a substrate. Examples of biomolecules useful herein include, but are not limited to, an antibody, a peptide, a small molecule, a lectin, a modified polysaccharide, a synthetic composite macromolecule, a functionalized nanostructure, a synthetic polymer, a modified/blocked nucleotides/nucleoside, a modified/blocked amino acid, a fluorophore, a chromophore, a ligand, a chelate, or a hapten.

In one aspect, the biomolecule comprises a drug such as a small molecule. In one aspect, any drug that interacts with DNA inside a cell (e.g., induce supercoiling or condensation) can be used. Examples of drugs useful herein include, but are not limited to, spermine, spermidine, or poly-lysine.

In one aspect, the biomolecule can be a protein. For example, the protein can include peptides, fragments of proteins or peptides, membrane-bound proteins, or nuclear proteins. The protein can be of any length, and can include one or more amino acids or variants thereof. The protein(s) can be fragmented, such as by protease digestion, prior to analysis. A protein sample to be analyzed can also be subjected to fractionation or separation to reduce the complexity of the samples. Fragmentation and fractionation can also be used together in the same assay. Such fragmentation and fractionation can simplify and extend the analysis of the proteins.

In another aspect, the biomolecule is a virus. Examples of viruses include, but are not limited to, Herpes simplex virus type-1, Herpes simplex virus type-2, Cytomegalovirus, Epstein-Barr virus, Varicella-zoster virus, Human herpesvirus 6, Human herpesvirus 7, Human herpesvirus 8, Variola virus, Vesicular stomatitis virus, Hepatitis A virus, Hepatitis B virus, Hepatitis C virus, Hepatitis D virus, Hepatitis E virus, Rhinovirus, Coronavirus, Influenza virus A, Influenza virus B, Measles virus, Polyomavirus, Human Papilomavirus, Respiratory syncytial virus, Adenovirus, Coxsackie virus, Dengue virus, Mumps virus, Poliovirus, Rabies virus, Rous sarcoma virus, Yellow fever virus, Ebola virus, Marburg virus, Lassa fever virus, Eastern Equine Encephalitis virus, Japanese Encephalitis virus, St. Louis Encephalitis virus, Murray Valley fever virus, West Nile virus, Rift Valley fever virus, Rotavirus A, Rotavirus B, Rotavirus C, Sindbis virus, Simian Immunodeficiency cirus, Human T-cell Leukemia virus type-1, Hantavirus, Rubella virus, Simian Immunodeficiency virus, Human Immunodeficiency virus type-1, Vaccinia virus, SARS virus, Human Immunodeficiency virus type-2, leutivirus, adeno-associated bacterivirus, or any strain or variant thereof.

“Aptamers” are also contemplated herein and are molecules that interact with a target molecule, preferably in a specific way. Typically aptamers are small nucleic acids ranging from 15-50 bases in length that fold into defined secondary and tertiary structures, such as stem-loops or G-quartets. Aptamers can bind small molecules, such as ATP (U.S. Pat. No. 5,631,146) and theophiline (U.S. Pat. No. 5,580,737), as well as large molecules, such as reverse transcriptase (U.S. Pat. No. 5,786,462) and thrombin (U.S. Pat. No. 5,543,293). Representative examples of how to make and use aptamers to bind a variety of different target molecules can be found in the following non-limiting list of U.S. Pat. Nos. 5,476,766, 5,503,978, 5,631,146, 5,731,424, 5,780,228, 5,792,613, 5,795,721, 5,846,713, 5,858,660, 5,861,254, 5,864,026, 5,869,641, 5,958,691, 6,001,988, 6,011,020, 6,013,443, 6,020,130, 6,028,186, 6,030,776, and 6,051,698, which are incorporated by reference herein for at least their teachings of aptamers.

“Ribozymes” are also contemplated herein and are nucleic acid molecules that are capable of catalyzing a chemical reaction, either intramolecularly or intermolecularly. Ribozymes are thus catalytic nucleic acid. There are a number of different types of ribozymes that catalyze nuclease or nucleic acid polymerase type reactions which are based on ribozymes found in natural systems, such as hammerhead ribozymes, (for example, but not limited to the following U.S. Pat. Nos. 5,334,711, 5,436,330, 5,616,466, 5,633,133, 5,646,020, 5,652,094, 5,712,384, 5,770,715, 5,856,463, 5,861,288, 5,891,683, 5,891,684, 5,985,621, 5,989,908, 5,998,193, 5,998,203, WO 9858058 by Ludwig and Sproat, WO 9858057 by Ludwig and Sproat, and WO 9718312 by Ludwig and Sproat) hairpin ribozymes (for example, but not limited to the following U.S. Pat. Nos. 5,631,115, 5,646,031, 5,683,902, 5,712,384, 5,856,188, 5,866,701, 5,869,339, and 6,022,962), and tetrahymena ribozymes (for example, but not limited to the following U.S. Pat. Nos. 5,595,873 and 5,652,107). There are also a number of ribozymes that are not found in natural systems, but which have been engineered to catalyze specific reactions de novo (for example, but not limited to the following U.S. Pat. Nos. 5,580,967, 5,688,670, 5,807,718, and 5,910,408). Ribozymes typically cleave nucleic acid substrates through recognition and binding of the target substrate with subsequent cleavage. This recognition is often based mostly on canonical or non-canonical base pair interactions. This property makes ribozymes particularly good candidates for target specific cleavage of nucleic acids because recognition of the target substrate is based on the target substrates sequence. Representative examples of how to make and use ribozymes to catalyze a variety of different reactions can be found in the following non-limiting list of U.S. Pat. Nos. 5,646,042, 5,693,535, 5,731,295, 5,811,300, 5,837,855, 5,869,253, 5,877,021, 5,877,022, 5,972,699, 5,972,704, 5,989,906, and 6,017,756. These patents are all incorporated by reference herein at least for their teachings of ribozymes.

In one aspect, the biomolecule comprises a nucleic acid. The nucleic acid can be an oligonucleotide, deoxyribonucleic acid (DNA) (single or double stranded), ribonucleic acid (RNA) (single or double stranded), or peptide nucleic acid (PNA). The nucleic acid can be nucleic acid from any source, such as a nucleic acid obtained from cells in which it occurs in nature, recombinantly produced nucleic acid, or chemically synthesized nucleic acid. For example, the nucleic acid can be cDNA or genomic DNA or DNA synthesized to have the nucleotide sequence corresponding to that of naturally-occurring DNA. The nucleic acid can also be a mutated or altered form of nucleic acid (e.g., DNA that differs from a naturally occurring DNA by an alteration, deletion, substitution or addition of at least one nucleic acid residue) or nucleic acid that does not occur in nature.

In one aspect, the nucleic acid can be present in a vector such as an expression vector (e.g., a plasmid or viral-based vector). In another aspect, the nucleic acid selected can be introduced into cells in such a manner that it becomes integrated into genomic DNA and is expressed or remains extrachromosomal (i.e., is expressed episomally). In another aspect, the vector is a chromosomally integrated vector. The nucleic acids useful herein can be linear or circular and can be of any size. In one aspect, the nucleic acid can be single or double stranded DNA or RNA.

In one aspect, the biomolecule is a nucleic acid that inhibits a function of a gene in the cell. In one aspect, small gene fragments that encode dominant-acting synthetic genetic elements (SGEs), e.g., molecules that interfere with the function of genes from which they are derived (antagonists) or that are dominant constitutively active fragments (agonists) of such genes, can be used as the biomolecule. SGEs that can be identified by the subject method include, but are not limited to, polypeptides, inhibitory antisense RNA molecules, ribozymes, nucleic acid decoys, and small peptides.

The SGEs identified by the present method may function to inhibit the function of an endogenous gene at the level of nucleic acids, e.g., by an antisense or decoy mechanism, or by encoding a polypeptide that is inhibitory through a mechanism of interference at the protein level, e.g., a dominant negative fragment of the native protein. Alternatively, certain SGEs can function to potentiate (including mimicking) the function of an endogenous gene by encoding a polypeptide which retains at least a portion of the bioactivity of the corresponding endogenous gene, and may in particular instances be constitutively active. The small gene fragments and SGE libraries disclosed in U.S. Patent Publication No. 2003/0228601, which is incorporated by reference in its entirety, can be used herein.

In one aspect, the biomolecule is an RNAi agent. An RNAi agent is an agent that modulates expression of a target gene by a RNA interference mechanism. In one aspect, the RNAi agent can be small ribonucleic acid molecules (also referred to herein as interfering ribonucleic acids), i.e., oligoribonucleotides, that are present in duplex structures, e.g., two distinct oligoribonucleotides hybridized to each other or a single ribooligonucleotide that assumes a small hairpin formation to produce a duplex structure. By oligoribonucleotide is meant a ribonucleic acid that does not exceed about 100 nt in length, and typically does not exceed about 75 nt length, where the length in certain embodiments is less than about 70 nt. When the RNAi agent is a duplex structure of two distinct ribonucleic acids hybridized to each other, e.g., an siRNA, such as d-siRNA, the length of the duplex structure typically ranges from about 15 to 30 bp, usually from about 15 to 29 bp, where lengths between about 20 and 29 bps, e.g., 21 bp, 22 bp, can be used. Where the RNAi agent is a duplex structure of a single ribonucleic acid that is present in a hairpin formation, i.e., a shRNA, the length of the hybridized portion of the hairpin is typically the same as that provided above for the siRNA type of agent or longer by 4-8 nucleotides. The weight of the RNAi agents of this embodiment typically ranges from about 5,000 daltons to about 35,000 daltons, and in many embodiments is at least about 10,000 daltons and less than about 27,500 daltons, often less than about 25,000 daltons.

In certain aspects, instead of the RNAi agent being an interfering ribonucleic acid, e.g., an siRNA or shRNA as described above, the RNAi agent can encode an interfering ribonucleic acid, e.g., an shRNA, as described above. In other words, the RNAi agent can be a transcriptional template of the interfering ribonucleic acid. In these aspects, the transcriptional template can be a DNA that encodes the interfering ribonucleic acid. The RNAi agents disclosed in International Publication No. WO2004/0798950, which is incorporated by reference in its entirety, can be used herein.

In one aspect, when then biomolecule is DNA or RNA, the amount of DNA or RNA present in the spotting solution is from 0.125 mg/mL to 2 mg/mL. In another aspect, the amount of DNA or RNA is 0.125 mg/mL, 0.2 mg/mL, 0.3 mg/mL, 0.4 mg/mL, 0.5 mg/mL, 0.6 mg/mL, 0.7 mg/mL, 0.8 mg/mL, 0.9 mg/mL, 1.0 mg/mL, 1.2 mg/mL, 1.3 mg/mL, 1.4 mg/mL, 1.5 mg/mL, 1.6 mg/mL, 1.7 mg/mL, 1.8 mg/mL, 1.9 mg/mL, or 2.0 mg/mL, where any concentration value can form a lower and upper endpoint of a concentration range. In one aspect, when the biomolecule is DNA, the DNA has a length of from 25 mer to 2,500 mer. In another aspect, the length of the DNA is 25 mer, 50 mer, 100 mer, 200 mer, 300 mer, 400 mer, 500 mer, 600 mer, 700 mer, 800 mer, 900 mer, 1,000 mer, 1,200 mer, 1,400 mer, 1,600 mer, 1,800 mer, 2,000 mer, 2,200 mer, 2,400 mer, or 2,500 mer, where any length can be a lower or upper endpoint of a DNA length range.

Described herein are methods for depositing a biomolecule on a support comprising

(a) providing a composition comprising an alkylene diol, a betaine, a detergent, a salt, and the biomolecule; and

(b) depositing the solution on the support to produce a spot comprising the biomolecule on the surface of the support.

In one aspect, the depositing step comprises immersing the tip of a pin into the composition comprising the biomolecule; removing the tip from the composition, wherein the tip comprises the composition; and transferring the composition to the support. This aspect can be accomplished, for example, by using a typographic pin array. The depositing step can be carried out using an automated, robotic printer. Such robotic systems are available commercially from, for example, Intelligent Automation Systems (IAS), Cambridge, Mass.

The pin can be solid or hollow. The tips of solid pins are generally flat, and the diameter of the pins determines the volume of fluid that is transferred to the substrate. Solid pins having concave bottoms can also be used. In one aspect, to permit the printing of multiple arrays with a single sample loading, hollow pins that hold larger sample volumes than solid pins and therefore allow more than one array to be printed from a single loading can be used. Hollow pins include printing capillaries, tweezers and split pins. An example of a preferred split pen is a micro-spotting pin that TeleChem International (Sunnyvale, Calif.) has developed. In one aspect, pins made by Point Tech can be used herein. The spotting solutions described herein can be used in a number of commercial spotters including, but not limited to, Genetix and Biorobotics spotters.

According to the method, any solid support may be employed, so long as it is capable of retaining the printed biomolecule. In one aspect, the solid support has a planar surface upon which the biomolecule is deposited. The supports that can be used herein include, but are not limited to, a microplate, a slide, or any other material that can support cell growth. In one aspect, when the substrate is a microplate, the number of wells and well volume will vary depending upon the scale and scope of the analysis. Other examples of substrates useful herein include, but are not limited to, a cell culture surface such as a 24-well dish, 8-well dish, 10 cm dish, or a T75 flask. In one aspect, the support comprises one or more biomolecules (e.g., nucleic acids having a known sequence), wherein the support (e.g., an array) has a plurality of locations, wherein the support comprises at least 96 or 192 locations. In one aspect, the locations can be from 200 to 500 μm apart from each other. In one aspect, a typographical pin array having a matrix of pins aligned such that each pin from the matrix fits into a corresponding source well, e.g., a well from a microtiter plate, can be used. In this aspect, the pin array can also be used in conjunction with a redrawn capillary-imaging reservoir. See International Patent Application WO 99/55460, incorporated herein by reference. Methods for the fabrication and use of high-density nucleic acid arrays are set forth in Microarray Biochip Technology, M. Schena, ed. Eaton Publishing, Natick, Mass. (2000).

For optical or electrical detection applications, the support can be transparent, impermeable, or reflecting, as well as electrically conducting, semiconducting, or insulating. For biological applications, the support material can be either porous or nonporous and can be selected from either organic or inorganic materials.

In one aspect, the support comprises a plastic, a polymeric or co-polymeric substance, a ceramic, a glass, a metal, a crystalline material, a noble or semi-noble metal, a metallic or non-metallic oxide, a transition metal, or any combination thereof. Additionally, the support can be configured so that it can be placed in any detection device. In one aspect, sensors can be integrated into the bottom/underside of the support and used for subsequent detection. These sensors could include, but are not limited to, optical gratings, prisms, electrodes, and quartz crystal microbalances. Detection methods could include fluorescence, phosphorescence, chemiluminescence, refractive index, mass, electrochemical. In one aspect, the support is a Corning LID microplate.

In another aspect, the support comprises a porous, inorganic layer. Any of the porous supports and methods of making such supports disclosed in U.S. Pat. No. 6,750,023, which is incorporated by reference in its entirety, can be used herein. In one aspect, the inorganic layer on the support comprises a glass or metal oxide. In another aspect, the inorganic layer comprises a silicate, an aluminosilicate, a boroaluminosilicate, a borosilicate glass, or a combination thereof. In a further aspect, the inorganic layer comprises TiO₂, SiO₂, Al₂O₃, Cr₂O₃, CuO, ZnO, Ta₂O₅, Nb₂O₅, ZnO₂, or a combination thereof.

In another aspect, the support can be composed of an organic material. Organic materials useful herein can be made from polymeric materials due to their dimensional stability and resistance to solvents. Examples of organic support materials include, but are not limited to, polyesters, such as polyethylene terephthalate and polybutylene terephthalate; polyvinylchloride; polyvinylidene fluoride; polytetrafluoroethylene; polycarbonate; polyamide; poly(meth)acrylate; polystyrene, polyethylene; or ethylene/vinyl acetate copolymer.

In one aspect, the support can be composed of an inorganic material. Examples of inorganic support materials include, but are not limited to, metals, semiconductor materials, glass, and ceramic materials. Examples of metals that can be used as support materials include, but are not limited to, gold, platinum, nickel, palladium, aluminum, chromium, steel, and gallium arsenide. Semiconductor materials used for the support material include, but are not limited to, silicon and germanium. Glass and ceramic materials used for the support material can include, but are not limited to, quartz, glass, porcelain, alkaline earth aluminoborosilicate glass and other mixed oxides. Further examples of inorganic support materials include graphite, zinc selenide, mica, silica, lithium niobate, and inorganic single crystal materials.

In one aspect, when the support is a glass, the support is a two-dimensional solid glass surface, such as commercially available glass microscope slides (3″×1″) made of soda lime, or other glass compositions. In one aspect, the glass support can be made from either a boroaluminosilicate or a borosilicate glass. Other glass supports can include three-dimensional porous glass surfaces or porous glass supports made by tape-cast or sol-gel processes from Pyrex™ glass frits. In one aspect, the glass support can have a surface that is functionalized or coated to facilitate the adhesion of the biomolecule. For instance, the surface can comprise one or more functional groups attached to the surface of the glass capable of forming a bond with the biomolecule. Examples of bonds that can be formed between the biomolecule and the support include, but are not limited to, covalent, electrostatic, ionic, hydrogen, or hydrophobic bonds. Examples of functional groups include, but are not limited to, amino, hydroxyl, or alkyl-thiol groups, acrylic acid, esters, anhydrides (e.g., styrene-co-maleic anhydride (SMA copolymer)), aldehyde, epoxide or other protected precursors capable of generating reactive functional groups. In one aspect, an aminating agent such as, for example, polylysine or an aminoalkylsilane, such as, for example, gamma-aminopropyl trimethoxysilane, N-(beta-aminoethyl)-gamma-aminopropyl trimethoxysilane, N-(beta-aminoethyl)-gamma-aminopropyl triethoxysilane or N′-(beta-aminoethyl)-gamma-aminopropyl methoxysilane) can be on the surface of the glass support.

In one aspect, after a solution comprising the biomolecule has been deposited on the support, the solution can be substantially (i.e., greater than 80%, greater than 85%, greater than 90%, greater than 95%, or greater than 99%) or completely removed to produce a dried spot on the support comprising the biomolecule. The drying temperature will vary depending upon the components used to produce the spotting solution. In one aspect, the spotting solution can be removed by evaporation of the solvent at room temperature from 1 hour to 24 hours, 1 hour to 22 hours, 1 hour to 20 hours, 1 hour to 18 hours, or 1 hour to 16 hours. Alternatively, heat can be applied to remove the solution. After removal of the solution (either substantial or complete), in one aspect, the dried spot of biomolecule has a diameter of from 50 μm to 150 μm. In another aspect, the spot has a diameter of 50 μm, 55 μm, 60 μm, 65 μm, 70 μm, 75 μm, 80 μm, 85 μm, 90 μm, 95 μm, 100 μm, 105 μm, 110 μm, 115 μm, 120 μm, 125 μm, 130 μm, 135 μm, 140 μm, 145 μm, or 150 μm, where any diameter size can be a lower and upper endpoint of a diameter range. In one aspect, greater than 80%, greater than 90%, or greater than 95% of the spots have a diameter of from 80 μm to 110 μm. In another aspect, greater than 80% of the spots, greater than 85% of the spots, greater than 90% of the spots, greater than 95% of the spots, greater than 99% of the spots, or 100% of the spots have the same diameter.

In one aspect, when the spotting solutions described herein are used to produce an array, the array can be interrogated using labeled targets (e.g., oligonucleotides, nucleic acid fragments such as cDNA and cRNA, PCR products, etc.). The targets can be labeled with fluorophores such as the Cy3, Cy5, or Alexa dyes, etc., or with other haptens such as biotin, digoxogenin. For example, the methods for biotinylating nucleic acids are familiar and described by Pierce (Avidin-Biotin Chemistry: A Handbook. Pierce Chemical Company, 1992, Rockford, Ill.).

Alternatively, in another aspect, to detect a hybridization event (i.e., the presence of the biotin), the solid support with the spotted biomolecule can be incubated with streptavidin/horseradish peroxidase conjugate. Such enzyme conjugates are commercially available from, for example, Vector Laboratories (Burlingham, Calif.). The streptavidin binds with high affinity to the biotin molecule bringing the horseradish peroxidase into proximity to the hybridized probe. Unbound streptavidin/horseradish peroxidase conjugate can be washed away in a simple washing step. The presence of horseradish peroxidase enzyme can then detected using a precipitating substrate in the presence of peroxide and the appropriate buffers. It is also possible to use chemiluminescent substrates for alkaline phosphatase or horseradish peroxidase (HRP), or fluorescence substrates for HRP or alkaline phosphatase. Examples include the diox substrates for alkaline phosphatase available from Perkin Elmer or Attophos HRP substrate from JBL Scientific (San Luis Obispo, Calif.).

The compositions and methods provide numerous advantages when compared to spotting solutions currently used. The compositions described herein can be used for a variety of printing pins. Furthermore, the compositions require fewer processing steps for the printing of biomolecules (e.g., nucleic acid microarrays). Additionally, the compositions produce consistently sized spots. This can be accomplished without a processing step that requires incubation at high humidity levels. Moreover, consistent spot morphology provides better contrast detection of the printed spot, and allows for easier optimization of printing parameters. The evaporation rate of the compositions is low, which is a desirable quality as it increases the length of time plates of biomolecule can be used before becoming empty. Conversely, current spotting solutions will have an increase in autofluorescence (i.e., background noise) as the ink ages, which reduces detection levels and sensitivity. Finally, the compositions described herein permit the long-term storage of biomolecules prior to depositing on a support, which addresses a requirement in this area of technology.

### EXAMPLES

The following examples are put forth so as to provide those of ordinary skill in the art with a complete disclosure and description of how the materials, articles, and methods described and claimed herein are made and evaluated, and are intended to be purely exemplary and are not intended to limit the scope of what the inventors regard as their invention. Efforts have been made to ensure accuracy with respect to numbers (e.g., amounts, temperature, etc.) but some errors and deviations should be accounted for. Unless indicated otherwise, parts are parts by weight, temperature is in ° C. or is at ambient temperature, and pressure is at or near atmospheric. There are numerous variations and combinations of reaction conditions, e.g., component concentrations, desired solvents, solvent mixtures, temperatures, pressures and other reaction ranges and conditions that can be used to optimize the product purity and yield obtained from the described process. Only reasonable and routine experimentation will be required to optimize such process conditions.

### Example 1

The spotting composition was prepared by the following ingredients: (all are listed in final percentages):

50% ethylene glycol

2×SSC (0.3 M sodium chloride, 0.03 M sodium citrate)

0.5 M Betaine

0.07% caprylic acid

0.05 M NaOH

The following amounts of each component was used to make a 100 milliliter batch of spotting solution:

28.3 ml of 18 mega-ohm distilled water

50 ml of 100% Ethylene Glycol

10 ml of 20×SSC (3 M sodium chloride, 0.3 M sodium citrate) buffer

10 ml of 5 molar Betaine

0.7 ml of 10% w/v caprylic acid

1.0 ml of 5 N sodium hydroxide

100 ml total volume

The final pH of this ink was between 11 and 12. The ingredients were added in the order listed, mixed well, then ready for use. The spotting solution can be made to any volume desired if concentrations of each component remain constant. By varying the amount of sodium hydroxide that is added, it is possible alter the pH of the composition.

### Example 2

### Use of Spotting Solution

To use the spotting solution listed above for DNA, DNA in either oligonucleotide or full-length form was dried down completely, and then resuspended in the desired volume of the spotting solution of Example 1. Once this is done, the DNA-spotting solution is ready for use (e.g., in a microarray printer). Hybridization of DNA was performed using Pronto!™ Universal Microarray Kits (www.coming.com/lifesciences/pronto).

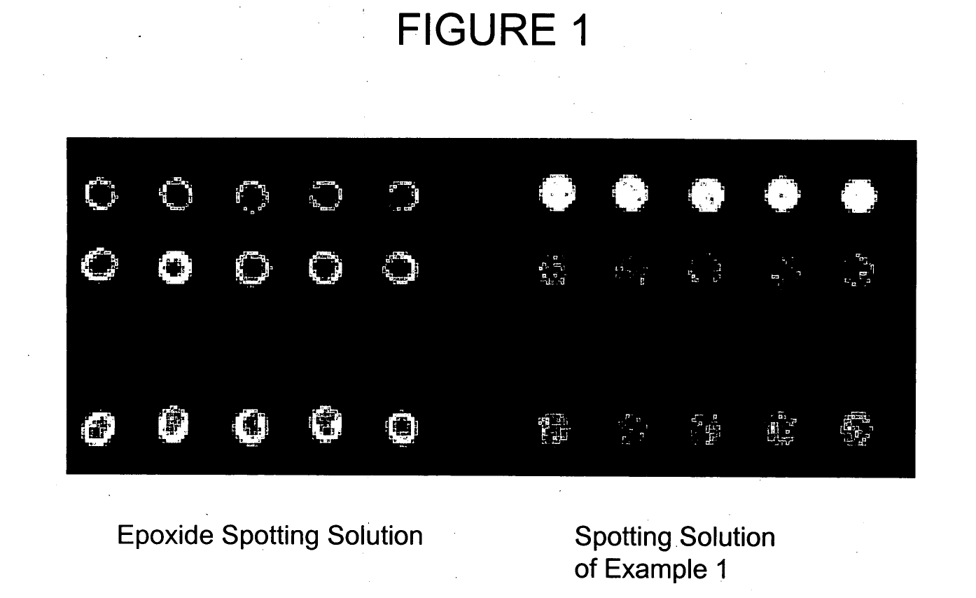

FIG. 1 shows the hybridization of an oligo array with Point Tech pins. The Corning epoxide spotting solution (phosphates, ethylene glycol, SDS, dextran) produced spots with low intensity in the center of each spot. The spots produced by the solution of Example 1 have uniform signals. The spotting solution of Example 1 provides predictable spot morphology, which is useful when it comes to producing large arrays.

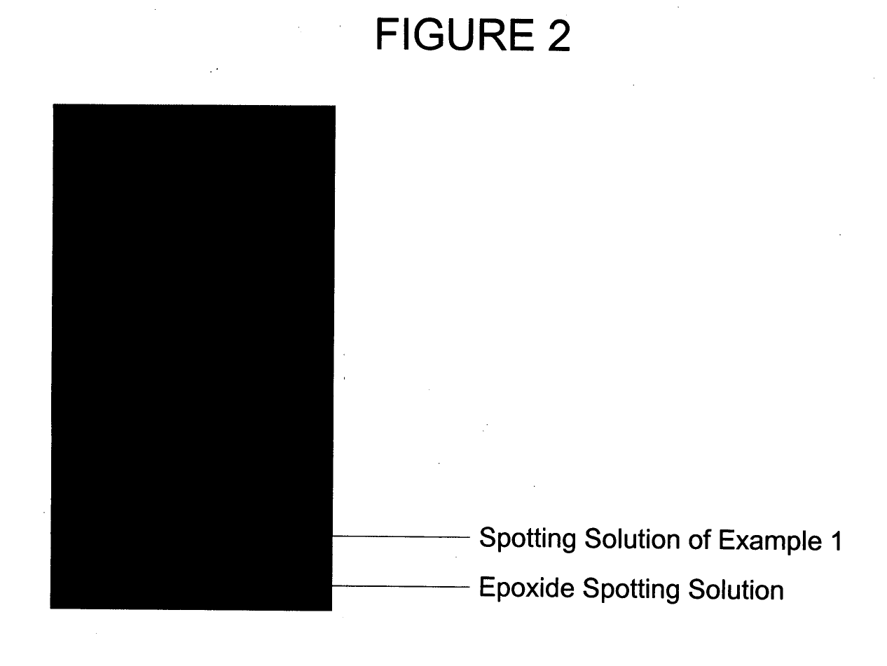

FIG. 2 shows ink-only spots. The Corning epoxide spotting solution produced a detectable signal, whereas the spotting solution of Example 1 produced no detectable signal. It is useful for the spotting solution not to produce a detectable signal so that it does not contribute to the S/N ratio or, in the alternative, produce a false result. For example, the Corning epoxide solution could suggest that DNA was present when it is not.

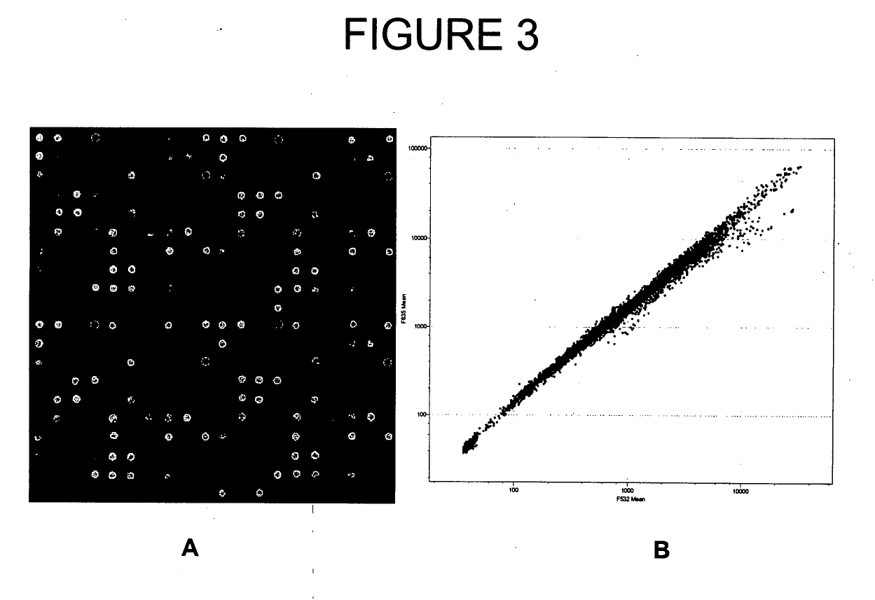

FIG. 3 shows oligos spotted with the solution of Example 1 followed by hybridization cDNA and scanned with Cy3/Cy5 PMT showed no irregularities. Illumina oligos were dissolved in the spotting solution of Example 1 and spotted on Epoxide Coated Slides using a Genetix printer fitted with 75-μm PointTech pins. The arrays were hybridized with cDNA made from Human Universal Reference RNA, Pronto!™ hybridization reagents, and scanned at 550/600 Cy3/Cy5 PMT. FIG. 3B shows no irregularities after hybridization, as indicated by the relatively straight line.

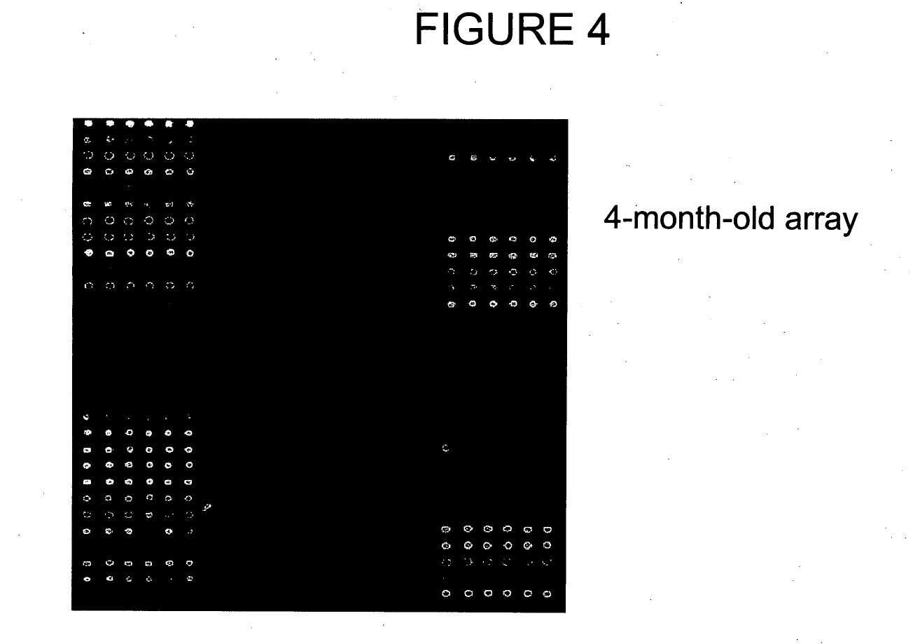

FIG. 4 shows the ability of arrays produced from the spotting solution of Example 1 can be stored at least for four months without reduction of signal produced by the spotting solution.

Throughout this application, various publications are referenced. The disclosures of these publications in their entireties are hereby incorporated by reference into this application in order to more fully describe the compounds, compositions and methods described herein.

Various modifications and variations can be made to the materials, methods, and articles described herein. Other aspects of the materials, methods, and articles described herein will be apparent from consideration of the specification and practice of the materials, methods, and articles disclosed herein. It is intended that the specification and examples be considered as exemplary.

What is claimed:

1. A composition comprising
(a) an alkylene diol;
(b) a betaine;
(c) a detergent; and
(d) a salt.

2. The composition of claim 1, wherein the alkylene diol comprises the formula HO(CH₂)ₙOH, wherein n is an integer of from 1 to 25.

3. The composition of claim 1, wherein the alkylene diol comprises methylene glycol, ethylene glycol, propylene glycol, butylene glycol, or a mixture thereof.

4. The composition of claim 1, wherein the alkylene diol comprises ethylene glycol.

5. The composition of claim 1, wherein the alkylene diol is from 30 to 70% by volume of the composition.

6. The composition of claim 1, wherein the alkylene diol is from 40 to 60% by volume of the composition.

7. The composition of claim 1, wherein the betaine comprises an alkylbetaine, an amidobetaine, a sulfobetaine, or a phosphobetaine.

8. The composition of claim 1, wherein the betaine comprises the formula (R¹)(R²)(R³)N⁺—CH₂—COO⁻, wherein R¹, R², and R³ are, independently, hydrogen or an alkyl group, wherein the betaine is a neutral compound or the salt thereof.

9. The composition of claim 8, wherein R¹, R², and R³ are, independently, methyl, ethyl, propyl, butyl, or pentyl.

10. The composition of claim 8, wherein R¹, R², and R³ are methyl.

11. The composition of claim 1, wherein the detergent comprises an anionic surfactant.

12. The composition of claim 11, wherein the anionic surfactant comprises an alkyl aryl sulfonate, an alkyl sulfate, or sulfated oxyethylated alkyl phenol.

13. The composition of claim 11, wherein the anionic surfactant comprises sodium dodecylbenzene sulfonate, sodium decylbenzene sulfonate, ammonium methyl dodecylbenzene sulfonate, ammonium dodecylbenzene sulfonate, sodium octadecylbenzene sulfonate, sodium nonylbenzene sulfonate, sodium dodecylnaphthalene sulfonate, sodium hetadecylbenzene sulfonate, potassium eicososyl naphthalene sulfonate, ethylamine undecylnaphthalene sulfonate, sodium docosylnaphthalene sulfonate, sodium octadecyl sulfate, sodium hexadecyl sulfate, sodium dodecyl sulfate, sodium nonyl sulfate, ammonium decyl sulfate, potassium tetradecyl sulfate, diethanolamino octyl sulfate, triethanolamine octadecyl sulfate, amrnmonium nonyl sulfate, ammonium nonylphenoxyl tetraethylenoxy sulfate, sodium dodecylphenoxy triethyleneoxy sulfate, ethanolamine decylphenoxy tetraethyleneoxy sulfate, or potassium octylphenoxy triethyleneoxy sulfate.

14. The composition of claim 1, wherein the detergent comprises a nonionic surfactant.

15. The composition of claim 14, wherein the nonionic surfactant comprises the condensation product between ethylene oxide or propylene oxide with the propylene glycol, ethylene diamine, diethylene glycol, dodecyl phenol, nonyl phenol, tetradecyl alcohol, N-octadecyl diethanolamide, N-dodecyl monoethanolamide, polyoxyethylene sorbitan monooleate, or polyoxyethylene sorbitan monolaurate.

16. The composition of claim 1, wherein the detergent comprises a cationic surfactant.

17. The composition of claim 16, wherein the cationic surfactant comprises ethyl-dimethylstearyl ammonium chloride, benzyl-dimethyl-stearyl ammonium chloride, benzyldimethyl-stearyl ammonium chloride, trimethyl stearyl ammonium chloride, trimethylcetyl ammonium bromide, dimethylethyl dilaurylammonium chloride, dimethyl-propyl-myristyl ammonium chloride, or the corresponding methosulfate or acetate.

18. The composition of claim 1, wherein the detergent comprises an organic acid or the salt thereof.

19. The composition of claim 1, wherein the detergent comprises the formula CH₃(CH₂)ₘCO₂H, wherein m is from 1 to 25, or the salt thereof.

20. The composition of claim 1, wherein the detergent comprises hexanoic acid, heptenoic acid, octanoic acid, nonanoic acid, decanoic acid, or the salt thereof.

21. The composition of claim 1, wherein the detergent comprises octanoic acid or the salt thereof.

22. The composition of claim 1, wherein the salt comprises an organic salt, an inorganic salt, or a mixture thereof.

23. The composition of claim 22, wherein the organic salt comprises a citrate.

24. The composition of claim 22, wherein the inorganic salt comprises NaCl, KCl, MgCl₂, LiCl, or a mixture thereof.

25. The composition of claim 1, wherein the salt comprises a mixture of NaCl and sodium citrate.

26. The composition of claim 1, wherein the composition comprises an alkaline pH.

27. The composition of claim 26, wherein the pH is greater than 8.5.

28. The composition of claim 1, wherein the composition further comprises water.

29. The composition of claim 1, wherein the composition further comprises a biomolecule.

30. The composition of claim 29, wherein the biomolecule comprises a nucleic acid, an antibody, a peptide, a small molecule, a lectin, a modified polysaccharide, a synthetic composite macromolecule, a functionalized nanostructure, a synthetic polymer, a modified/blocked nucleotides/nucleoside, a modified/blocked amino acid, a fluorophore, a chromophore, a ligand, a chelate, or a hapten.

31. The composition of claim 29, wherein the biomolecule comprises DNA<RNA, or an oligonucleotide.

32. The composition of claim 1, wherein the alkylene diol is ethylene glycol, the betaine is Me₃N⁺—CH₂—COO⁻, the detergent is octanoic acid, and the salt is NaCl and sodium citrate.

33. The composition of claim 32, wherein the composition further comprises water, and the pH is greater than or equal to 8.5.

34. The composition of claim 33, wherein the composition further comprises a nucleic acid.

35. The composition of claim 34, wherein the nucleic acid comprises an oligonucleotide.

36. The composition of claim 34, wherein the nucleic acid comprises single-stranded DNA or RNA or double-stranded DNA or RNA.

37. The composition of claim 36, wherein the amount of DNA or RNA is from 0.125 mg/mL to 2 mg/mL.

38. The composition of claim 34, wherein the nucleic acid is DNA having a length of from 25 mer to 2,500 mer.

39. A method for making a composition comprising admixing an alkylene diol, a betaine; a detergent; and a salt.

40. A composition made by the method of claim 39.

41. A method for depositing a biomolecule on a support comprising
(a) providing a composition comprising an alkylene diol, a betaine, a detergent, a salt, and the biomolecule; and
(b) depositing the solution on the support to produce a spot comprising the biomolecule on the surface of the support.

42. The method of claim 41, wherein the depositing step comprises immersing the tip of a pin into the composition comprising the biomolecule; removing the tip from the composition, wherein the tip comprises the composition; and transferring the composition to the support.

43. The method of claim 41, wherein the depositing step is repeated a plurality of times to provide an array of one or more biomolecules.

44. The method of claim 41, wherein the support comprises a plastic, a polymeric or co-polymeric substance, a ceramic, a glass, a metal, a crystalline material, a noble or semi-noble metal, a metallic or non-metallic oxide, a transition metal, or any combination thereof.

45. The method of claim 41, wherein the support comprises a glass comprising one or more functional groups attached to the surface of the glass capable of forming a bond with the biomolecule.

46. The method of claim 45, wherein the functional group comprises an amino group, a hydroxyl group, an alkyl-thiol group, an acrylic acid, an ester, an anhydride, an aldehyde, or an epoxide.

47. The method of claim 45, wherein the functional group comprises an epoxy group.

48. The method of claim 41, wherein the alkylene diol comprises the formula HO(CH₂)ₙOH, wherein n is an integer of from 1 to 25.

49. The method of claim 41, wherein the alkylene diol comprises methylene glycol, ethylene glycol, propylene glycol, butylene glycol, or a mixture thereof.

50. The method of claim 41, wherein the alkylene diol comprises ethylene glycol.

51. The method of claim 41, wherein the alkylene diol is from 30 to 70% by volume of the composition.

52. The method of claim 41, wherein the alkylene diol is from 40 to 60% by volume of the composition.

53. The method of claim 41, wherein the betaine comprises an alkylbetaine, an amidobetaine, a sulfobetaine, or a phosphobetaine.

54. The method of claim 41, wherein the betaine comprises the formula (R¹)(R²)(R³)N⁺—CH₂—COO⁻, wherein R¹, R², and R³ are, independently, hydrogen or an alkyl group, wherein the betaine is a neutral compound or the salt thereof.

55. The method of claim 54, wherein R¹, R², and R³ are, independently, methyl, ethyl, propyl, butyl, or pentyl.

56. The method of claim 54, wherein R¹, R², and R³ are methyl.

57. The method of claim 41, wherein the detergent comprises an anionic surfactant.

58. The method of claim 57, wherein the anionic surfactant comprises an alkyl aryl sulfonate, an alkyl sulfate, or sulfated oxyethylated alkyl phenol.

59. The method of claim 58, wherein the anionic surfactant comprises sodium dodecylbenzene sulfonate, sodium decylbenzene sulfonate, ammonium methyl dodecylbenzene sulfonate, ammonium dodecylbenzene sulfonate, sodium octadecylbenzene sulfonate, sodium nonylbenzene sulfonate, sodium dodecylnaphthalene sulfonate, sodium hetadecylbenzene sulfonate, potassium eicososyl naphthalene sulfonate, ethylamine undecylnaphthalene sulfonate, sodium docosylnaphthalene sulfonate, sodium octadecyl sulfate, sodium hexadecyl sulfate, sodium dodecyl sulfate, sodium nonyl sulfate, ammonium decyl sulfate, potassium tetradecyl sulfate, diethanolamino octyl sulfate, triethanolamine octadecyl sulfate, amrnmonium nonyl sulfate, ammonium nonylphenoxyl tetraethylenoxy sulfate, sodium dodecylphenoxy triethyleneoxy sulfate, ethanolamine decylphenoxy tetraethyleneoxy sulfate, or potassium octylphenoxy triethyleneoxy sulfate.

60. The method of claim 41, wherein the detergent comprises a nonionic surfactant.

61. The method of claim 60, wherein the nonionic surfactant comprises the condensation product between ethylene oxide or propylene oxide with the propylene glycol, ethylene diamine, diethylene glycol, dodecyl phenol, nonyl phenol, tetradecyl alcohol, N-octadecyl diethanolamide, N-dodecyl monoethanolamide, polyoxyethylene sorbitan monooleate, or polyoxyethylene sorbitan monolaurate.

62. The method of claim 41, wherein the detergent comprises a cationic surfactant.

63. The method of claim 62, wherein the cationic surfactant comprises ethyl-dimethylstearyl ammonium chloride, benzyl-dimethyl-stearyl ammonium chloride, benzyldimethyl-stearyl ammonium chloride, trimethyl stearyl ammonium chloride, trimethylcetyl ammonium bromide, dimethylethyl dilaurylammonium chloride, dimethyl-propyl-myristyl ammonium chloride, or the corresponding methosulfate or acetate.

64. The method of claim 41, wherein the detergent comprises an organic acid or the salt thereof.

65. The method of claim 41, wherein the detergent comprises the formula CH₃(CH₂)ₘCO₂H, wherein m is from 1 to 25, or the salt thereof.

66. The method of claim 41, wherein the detergent comprises hexanoic acid, heptenoic acid, octanoic acid, nonanoic acid, decanoic acid, or the salt thereof.

67. The method of claim 41, wherein the detergent comprises octanoic acid or the salt thereof.

68. The method of claim 41, wherein the salt comprises an organic salt, an inorganic salt, or a mixture thereof.

69. The method of claim 68, wherein the organic salt comprises a citrate.

70. The method of claim 68, wherein the inorganic salt comprises NaCl, KCl, MgCl₂, LiCl, or a mixture thereof.

71. The method of claim 41, wherein the salt comprises a mixture of NaCl and sodium citrate.

72. The method of claim 41, wherein the composition comprises an alkaline pH.

73. The method of claim 72, wherein the pH is greater than 8.5.

74. The method of claim 41, wherein the composition further comprises water.

75. The method of claim 41, wherein the biomolecule comprises a nucleic acid, a peptide, an antibody, a hapten, a biological ligand, a protein, a lipid, a small molecule, or a cell.

76. The method of claim 41, wherein the alkylene diol is ethylene glycol, the betaine is Me₃N⁺—CH₂—COO⁻, the detergent is octanoic acid, and the salt is NaCl, and sodium citrate.

77. The method of claim 76, wherein the composition further comprises water, and the pH is greater than or equal to 8.5.

78. The method of claim 77, wherein the composition further comprises a nucleic acid.

79. The method of claim 77, wherein the nucleic acid comprises an oligonucleotide.

80. The method of claim 77, wherein the nucleic acid comprises single-stranded DNA or RNA or double-stranded DNA or RNA.

81. The method of claim 80, wherein the amount of DNA or RNA is from 0.125 mg/mL to 2 mg/mL.

82. The method of claim 77, wherein the nucleic acid is DNA having a length of from 25 mer to 2,500 mer.

83. The method of claim 41, wherein after step (b), removing the solution to produce a dried spot comprising the biomolecule.

84. The method of claim 83, wherein the dried spot has a diameter of from 50 μm to 150 μm.

85. The method of claim 83, wherein the spot has a diameter of from 80 μm to 110 μm.
